(19) United States
(12) Patent Application Publication
(10) Pub. No.: US 2025/0237598 A1
(43) Pub. Date: Jul. 24, 2025

(54) APPARATUSES, METHODS AND COMPUTER PROGRAM PRODUCTS FOR IMAGING A STRUCTURE AT LEAST PARTIALLY OBSCURED, IN A VISIBLE WAVELENGTH RANGE, BY A LIQUID

(71) Applicant: Sony Group Corporation, Tokyo (JP)

(72) Inventors: Matthias SCHINZEL, Stuttgart (DE); Zoltan FACIUS, Stuttgart (DE)

(73) Assignee: Sony Group Corporation, Tokyo (JP)

(21) Appl. No.: 18/704,460

(22) PCT Filed: Oct. 21, 2022
(86) PCT No.: PCT/EP2022/079459

§ 371 Date: Apr. 25, 2024

(30) Foreign Application Priority Data
Nov. 2, 2021 (EP) 21206012.3

(51) Int. Cl. G01N 21/21; G01N 21/31; G01N 21/3577; G01N 21/55; G01N 33/14

(52) U.S. Cl. CPC G01N 21/21; G01N 21/314; G01N 21/3577; G01N 21/55; G01N 2021/3133; G01N 2021/3181; G01N 2021/556; G01N 33/146

(57) ABSTRACT

An apparatus for acquiring a wavelength and polarization state of light for imaging a structure at least partially obscured, in a visible wavelength range, by a liquid, is provided by the present disclosure.

### BACKGROUND

### Field of the Disclosure

The present invention relates to apparatuses, methods and computer program products for imaging a structure at least partially obscured, in a visible wavelength range, by a liquid.

### Description of the Related Art

The “background” description provided herein is for the purpose of generally presenting the context of the disclosure. Work of the presently named inventors, to the extent it is described in the background section, as well as aspects of the description which may not otherwise qualify as prior art at the time of filing, are neither expressly or impliedly admitted as prior art against the present invention.

In recent times, there has been significant development of image capture devices. Now, image capture devices are used in order to obtain images of objects and scenes in a wide range of different environments. Often, the images captured by these image capture devices are used in order to obtain information regarding the objects and scenes being imaged which can be used in order to assist a user or device (such as a robotic device) in performance of a given task.

For example, image capture devices may be used during a surgical operation or procedure in order to obtain images of a patient which can assist a person (such as a surgeon) in performing the operation. Moreover, imaging devices may be used in order to survey a scene or environment (as part of an engineering project or the like).

As such, optimized progression of a project or procedure may depend on the images which are obtained by the image capture devices (and the information which can be obtained from these images).

However, in some situations, it can be difficult to use the image capture devices in order to obtain images of a target object or structure within a scene. In particular, in some situations, other objects within the scene may obscure the target object or structure. In surgery, for example, a target object or structure may become obscured by certain liquids (such as blood). It becomes very difficult to obtain images of the target object or structure with an imaging device in these situations.

Accordingly, there is a desire to provide an apparatus which can provide images of a target object or structure within a scene.

It is an aim of the present disclosure to address these issues.

### SUMMARY

In a first aspect of the disclosure, an apparatus for acquiring a wavelength and polarization state of light for imaging a structure at least partially obscured, in a visible wavelength range, by a liquid, is provided, the apparatus comprising: a light source configured to generate light of a certain wavelength and polarization state, wherein the certain wavelength of light is a wavelength of light within a range of wavelengths outside a visible wavelength range; a beam splitting element to split light from a target, when the target is illuminated by the light source, along a first path and a second path; a polarization detection unit arranged on the first path of the beam splitting element and configured to measure a degree of polarization of light; an image capture device arranged on the second path of the beam splitting element and configured to measure intensity of light at wavelengths within the range of wavelengths outside the visible wavelength range; and circuitry configured to: control the light source to generate, in a sequence, light of at least one predetermined wavelength and polarization state to illuminate a liquid; and, for light of each of the at least one predetermined wavelength and polarization state, determine a level of scattering of the light by the liquid based on the degree of polarization of light measured by the polarization detection unit; determine a level of reflectance and absorption of the light by the liquid based on the intensity of light measured by the image capture device; and wherein the circuitry is further configured to acquire a wavelength and polarization state of light for imaging a structure at least partially obscured, in the visible wavelength range, by the liquid in accordance with the level of scattering, reflectance and absorption of light which has been determined for each of the at least one predetermined wavelength and polarization state.

In a second aspect of the disclosure, an apparatus for imaging a structure at least partially obscured, in a visible wavelength range, by a liquid, is provided, the apparatus comprising: a light source configured to illuminate the structure with light of a predetermined wavelength and polarization state, the predetermined wavelength and polarization state being acquired by an apparatus according to claim 1; and a second image capture device configured to generate a second image of the structure, as illuminated by the light source, at the predetermined wavelength.

In a third aspect of the disclosure, a method of acquiring a wavelength and polarization state of light for imaging a structure at least partially obscured, in a visible wavelength range, by a liquid, is provided, the method comprising: generating, using a light source, light of a certain wavelength and polarization state, wherein the certain wavelength of light is a wavelength of light within a range of wavelengths outside a visible wavelength range; splitting, using a beam splitting element, light from a target, when the target is illuminated by the light source, along a first path and a second path; measuring a degree of polarization of light using a polarization detection unit arranged on the first path of the beam splitting element; measuring intensity of light at wavelengths within the range of wavelengths outside the visible wavelength range using an image capture device arranged on the second path of the beam splitting element; controlling the light source to generate, in a sequence, light of at least one predetermined wavelength and polarization state to illuminate a liquid; and, for light of each of the at least one predetermined wavelength and polarization state, determining a level of scattering of the light by the liquid based on the degree of polarization of light measured by the polarization detection unit; determining a level of reflectance and absorption of the light by the liquid based on the intensity of light measured by the image capture device; acquiring a wavelength and polarization state of light for imaging a structure at least partially obscured, in the visible wavelength range, by the liquid in accordance with the level of scattering, reflectance and absorption of light which has been determined for each of the at least one predetermined wavelength and polarization state.

In a fourth aspect of the disclosure, a method of imaging a structure at least partially obscured, in a visible wavelength range, by a liquid, is provided, the method comprising: illuminating, using a light source, the structure with light of a predetermined wavelength and polarization state, the predetermined wavelength and polarization state being acquired by a method according to claim 14; and generating, using a second image capture device, a second image of the structure, as illuminated by the light source, at the predetermined wavelength.

Further embodiments of the present disclosure are defined by the appended claims.

According to embodiments of the disclosure, it is possible to obtain an image of a structure even when the structure is at least partially obscured, in the visible wavelength range, by a liquid. That is, in accordance with embodiments of the disclosure, a wavelength and polarization state of light can be acquired which enable an imaging device to see through the liquid thus providing an image with a better view of the structure beneath the liquid. This enables more detailed information regarding the scene to be obtained.

The advantageous technical effects provided by embodiments of the disclosure are not particularly limited to these advantageous technical effects. Further advantages associated with embodiments of the disclosure will become apparent to the skilled person when reading the description.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 illustrates an apparatus in accordance with embodiments of the disclosure;

FIG. 2 illustrates an example situation to which embodiments of the disclosure may be applied;

FIG. 3 illustrates an apparatus in accordance with embodiments of the disclosure;

FIG. 4 illustrates an example model of light transmission in accordance with embodiments of the disclosure;

FIG. 5A illustrates an example situation in accordance with embodiments of the disclosure;

FIG. 5B illustrates an example absorption spectra in accordance with embodiments of the disclosure;

FIG. 6A illustrates an example configuration of an apparatus in accordance with embodiments of the disclosure;

FIG. 6B illustrates an example configuration of an apparatus in accordance with embodiments of the disclosure;

FIG. 7 illustrates an example of scattering of light in accordance with embodiments of the disclosure;

FIG. 8 illustrates an example configuration of an apparatus in accordance with embodiments of the disclosure;

FIG. 9 illustrates a method in accordance with embodiments of the present disclosure;

FIG. 10 illustrates an example configuration of an apparatus in accordance with embodiments of the disclosure;

FIG. 11A illustrates an example image in accordance with embodiments of the disclosure;

FIG. 11B illustrates an example image in accordance with embodiments of the disclosure;

FIG. 11C illustrates an example image in accordance with embodiments of the disclosure;

FIG. 11D illustrates an example image in accordance with embodiments of the disclosure;

FIG. 12 disclosure illustrates an apparatus according to embodiments of the disclosure;

FIG. 13 illustrates an example colourization process in accordance with embodiments of the disclosure;

FIG. 14 illustrates an example colourization process in accordance with embodiments of the disclosure;

FIG. 15 illustrates a method in accordance with embodiments of the disclosure:

FIG. 16 illustrates an example endoscope system in accordance with embodiments of the disclosure.

### DESCRIPTION OF THE EMBODIMENTS

Referring now to the drawings, wherein like reference numerals designate identical or corresponding parts throughout the several views.

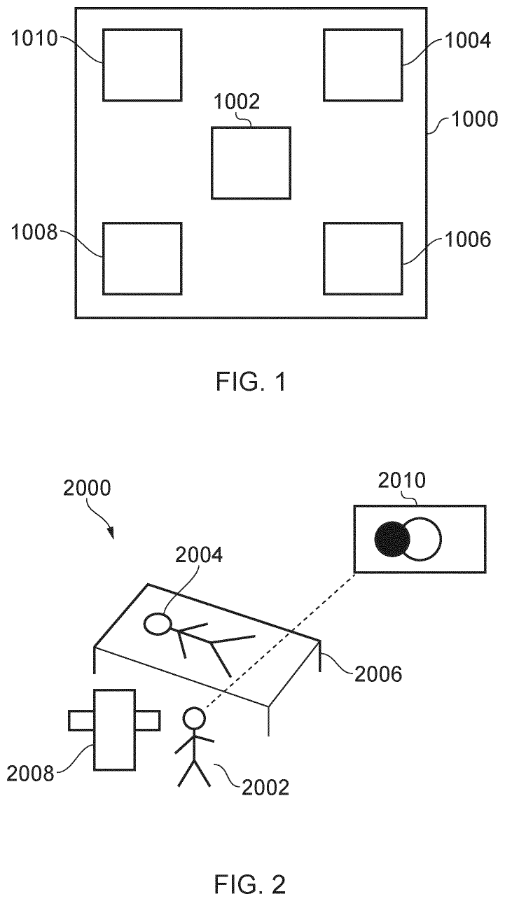

Referring to FIG. 1, an apparatus 1000 according to embodiments of the disclosure is shown. Typically, an apparatus 1000 according to embodiments of the disclosure is a computer device such as a personal computer or a terminal connected to a server. Indeed, in embodiments, the apparatus may also be a server. The apparatus 1000 is controlled using a microprocessor or other processing circuitry 1002. In some examples, the apparatus 1000 may be a portable computing device such as a mobile phone, laptop computer or tablet computing device.

The processing circuitry 1002 may be a microprocessor carrying out computer instructions or may be an Application Specific Integrated Circuit. The computer instructions are stored on storage medium 1004 which may be a magnetically readable medium, optically readable medium or solid state type circuitry. The storage medium 1004 may be integrated into the apparatus 1000 or may be separate to the apparatus 1000 and connected thereto using either a wired or wireless connection. The computer instructions may be embodied as computer software that contains computer readable code which, when loaded onto the processor circuitry 1002, configures the processor circuitry 1002 to perform a method according to embodiments of the disclosure.

Additionally, an optional user input device 1006 is shown connected to the processing circuitry 1002. The user input device 1006 may be a touch screen or may be a mouse or stylist type input device. The user input device 1006 may also be a keyboard or any combination of these devices.

A network connection 1008 may optionally be coupled to the processor circuitry 1002. The network connection 1008 may be a connection to a Local Area Network or a Wide Area Network such as the Internet or a Virtual Private Network or the like. The network connection 1008 may be connected to a server allowing the processor circuitry 1002 to communicate with another apparatus in order to obtain or provide relevant data. The network connection 1002 may be behind a firewall or some other form of network security.

Additionally, shown coupled to the processing circuitry 1002, is a display device 1010. The display device 1010, although shown integrated into the apparatus 1000, may additionally be separate to the apparatus 1000 and may be a monitor or some kind of device allowing the user to visualise the operation of the system. In addition, the display device 1010 may be a printer, projector or some other device allowing relevant information generated by the apparatus 1000 to be viewed by the user or by a third party.

Referring, now, to FIG. 2 of the present disclosure, an example situation in accordance with embodiments of the disclosure is illustrated.

In this specific example, a surgical operating theatre 2000 is shown.

A surgeon 2002 is present within the surgical operating theatre 2000. The surgeon 2002 is performing a surgical operation on a patient 2004. In this example, the patient 2004 is located on an operating table 2006.

Surgical equipment (not shown) may be provided within the surgical operating theatre 2000. This surgical equipment can be used by the surgeon during surgery (e.g. to perform a specific surgical task). This surgical equipment may include surgical tools such as forceps, scalpel, suction devices and the like. Furthermore, a number of other people may also be present within the operating theatre 2000 including, for example other medical personal who will assist the surgeon in performing surgery on the patient 2004.

In this example, the surgeon may also perform surgery on the patient 2004 with the assistance of a robotic surgical device 2008. The robotic surgical device may be a device which is under the control of the surgeon 2002 (such as a robotic arm) which can thus be used by the surgeon in order to perform at least a portion of the surgical operation on the patient. Alternatively, the robotic surgical device may be an autonomous or semi-autonomous robotic device which can perform at least a portion of the surgical operation on the patient under the supervision of the surgeon 2002.

An imaging device (not shown) may obtain images of the surgical scene (i.e. a portion of the patient being operated on) in order to assist the surgeon and/or the robot surgical device 2008 in their ability to perform the surgery. For example, the imaging device may image a certain tissue sample within the patient as the surgeon performs the surgical operation. In some examples, the imaging device may an surgical endoscope.

A display device 2010 may be provided in order to display the images of the surgical scene obtained by the imaging device (not shown) to the surgeon 2002. As such, the surgeon can view detailed images of the surgical scene (obtained by the imaging device) during the surgical procedure on the display device 2010.

However, during the performance of the surgical procedure, at least a portion of the tissue being operated on by the surgeon 2002 and/or the robotic surgical device 2008 may be covered by blood and/or other liquids or bodily fluids. The presence of this blood (or other liquids or bodily fluids) may prevent the surgeon 2002 and/or the robotic surgical device 2008 from being able to see the target tissue. As such, important information about the tissue (such as tissue anomalies or the like) may not be visible to the surgeon. This may include information about damaged tissue or information about other critical anatomic structures.

The surgeon (or other medical personal) may manually attempt to remove the blood (or other liquids or bodily fluids) from the surgical scene. In examples, manually attempting to remove the blood may include the use of a device such as a suction device. However, it can be very difficult to satisfactorily remove said liquids from the surgical scene in this manner. Firstly, the manual removal of the liquids from the surgical scene (using a suction device or the like) is only temporary, since new liquids may very quickly start to obscure the scene again. Moreover, the manual removal of the liquid from the scene may itself introduce a further problem or obstruction. For example, the use of a tool to remove the liquid (such as a suction device) may itself obscure the view of the scene and/or interfere with other tools being used by the surgeon. Alternatively, use of a tool to remove the liquid may also generate smoke within the surgical scene which will further obscure the view of the surgical scene.

Therefore, even when attempting to manually remove the blood (or other liquid) from the scene, a satisfactory image of the structure beneath the liquid may not be obtained.

Accordingly, during a surgical procedure, it can be very difficult to obtain an image of a target structure within the surgical scene, since the target structure may become at least partially obscured with a liquid.

For at least these reasons (and also the reasons as described in the Background) it is desired to provide a solution which enables images of a target object or structure within a scene to be obtained. Hence, an apparatus, method and computer program product are provided in accordance with the present disclosure, for acquiring a wavelength and polarization state of light for imaging a structure at least partially obscured, in a visible wavelength range, by a liquid.

### <Apparatus>

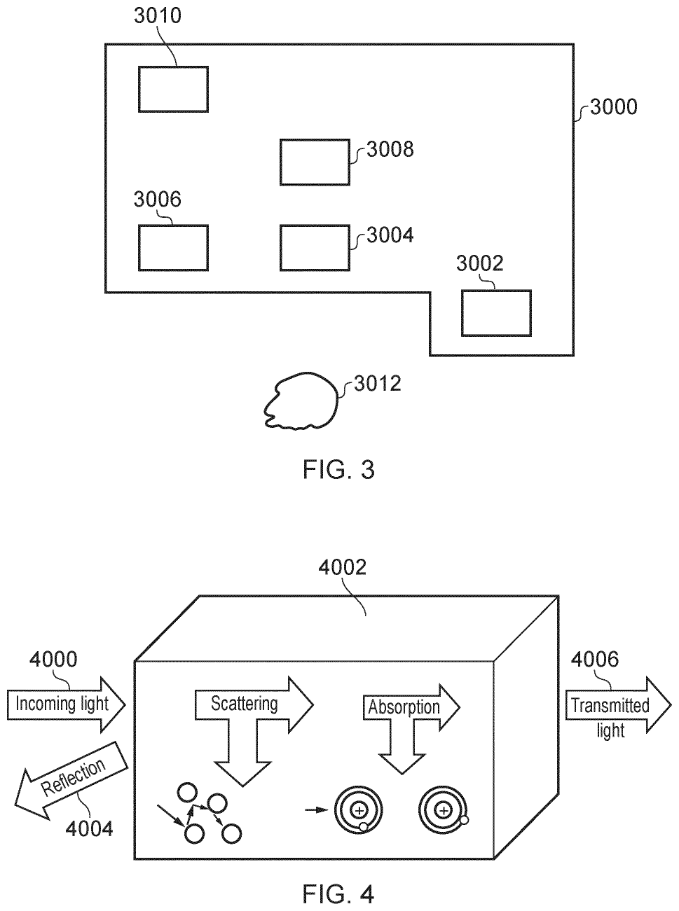

FIG. 3 illustrates an example configuration of an apparatus in accordance with embodiments of the disclosure. The apparatus 3000 illustrated in FIG. 3 is an apparatus for acquiring a wavelength and polarization state of light for imaging a structure at least partially obscured, in a visible wavelength range, by a liquid 3012. The apparatus 3000 comprises a light source 3002, a beam splitting element 3004, a polarization detection unit 3006, an image capture device 3008 and processing circuitry 3010. The processing circuitry 3010 may be processing circuitry such as processing circuitry 1002 described with reference to FIG. 1 of the present disclosure, for example.

The apparatus 3000 can be used in order to acquire a wavelength and polarization state of light for imaging a structure at least partially obscured, in a visible wavelength range, by a liquid. The acquired wavelength and polarization state of the light can then be used in order to see through the liquid such that an improved image of the structure can be obtained. Further details of apparatus 3000 will now be described.

### <Light Source>

Returning now to the example of FIG. 2 of the present disclosure, it will be appreciated that a liquid (such as blood) may be present within a surgical scene and may therefore obscure the image of a structure (such as target tissue) which can be obtained by an image capture device. This can prevent a surgeon from being able to see the structure, which may cause the surgeon difficulties in performing a surgical task.

In other words, the liquid may prevent light from the structure from reaching the imaging device such that the imaging device cannot obtain an image of the structure.

FIG. 4 illustrates an example model of light transmission in accordance with embodiments of the disclosure. In this example model, incoming light 4000 (from a light source) is shown. This light travels along a path until it encounters liquid 4002. When the light encounters the liquid 4002, a number of physical processes occur. Firstly, a portion of the incoming light 4000 is reflected off the surface of the liquid (the reflected portion 4004). However, some of the incoming light 4000 travels through the liquid 4002. As the incoming light 4000 travels through the liquid, a portion of that light is scattered off the particles of the liquid. Moreover, a portion of the light travelling through the liquid is absorbed by the particles of the liquid. Both the scattering and absorption of the light as it passes through the liquid attenuates the amount of light which passes through the liquid. Finally, a portion of the incoming light (the transmitted light 4006) is transmitted from the liquid. The amount of transmitted light 4006 is much less than the incoming light 4000 because of the physical processes of reflection, scattering and absorption which the incoming light experiences as it passes through the liquid.

If the incoming light is fully attenuated as it passes through the liquid then no light will be transmitted from the liquid. Accordingly, in this case, the liquid will be opaque to the incoming light. However, if only a portion of the incoming light is attenuated as it passes through the liquid (as illustrated in FIG. 4 of the present disclosure) then some light will be transmitted from the liquid and the liquid will appear translucent. However, if the incoming light is attenuated only a very small amount as it passes through the liquid (i.e. if the levels of reflectance, absorption and scattering are very low) then the liquid will appear transparent (such that the incoming light can be seen through the liquid).

Therefore, in order for a liquid to be transparent (such that it is possible to see incoming light through the liquid) then the liquid must have low reflectance, low absorption and low scattering of the light. If, however, the scattering is too high (for example) then the liquid will be translucent.

The processes of reflectance, absorption and scattering of the light by a liquid are wavelength and polarization dependent. In other words, a given liquid will reflect, absorb and scatter a different amount of the incoming light dependent on the wavelength and polarization state of the incoming light. Therefore, a liquid which is opaque at a first wavelength and polarization state of light may appear translucent or transparent at a different wavelength and polarization state of light.

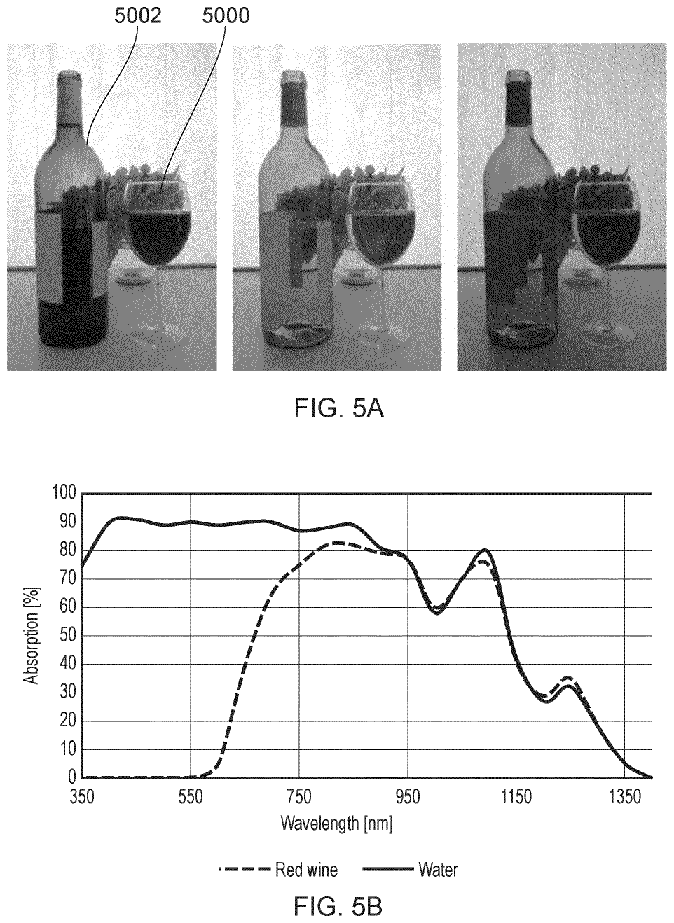

Consider, now, the example of FIG. 5A of the present disclosure. FIG. 5A illustrates an example situation in accordance with embodiments of the disclosure. Specifically, FIG. 5A demonstrates an example wavelength dependence of reflectance, absorption and scattering of a liquid.

In the first panel of FIG. 5A of the present disclosure, a scene is shown which comprise a glass 5000 and a bottle 5002. Each of the glass 5000 and the bottle 5002 are filled, at least partially, by a liquid. In this example, the liquid is red wine.

The image illustrated in the first panel of FIG. 5A of the present disclosure is an image taken by an image capture device sensitive to light of a visible wavelength range. Light of a visible wavelength range is defined as light which would be observable by a person. That is, the visible wavelength range is the segment of the electromagnetic spectrum which the human eye is capable of detecting. Typically, the visible wavelength range is defined as a wavelength range of 380 to 700 nm.

At this wavelength range (i.e. visible light) the red wine present in the glass 5000 and the bottle 5002 strongly attenuates the light by the processes of reflectance, absorption and scattering described with reference to FIG. 2 of the present disclosure. Hence, the wine in the glass 5000 and bottle 5002 appears almost opaque to visible light and thus obscures the view of any structure located behind the liquid.

FIG. 5B illustrates an example absorption spectra in accordance with embodiments of the disclosure. In particular, FIG. 5B illustrates an absorption spectra of red wine and water for a thickness of 1 cm.

The vertical axis of the absorption spectra defines the percentage attenuation of light by absorption, while the horizontal axis defines the wavelength of light.

In the visible wavelength range (e.g. 380 to 700 nm), the absorption of light by the red wine is very high (e.g. approximately 90%). As such, only a small amount of the light will be transmitted such that the liquid (red wine) will appear almost opaque at these wavelengths. This is why it is very difficult to see through a liquid such as red wine in the visible wavelength range. Indeed, many other liquids also have high absorption (low transmission) at these visible wavelengths. Indeed, taking surgery as an example, blood also has very high absorption (low transmission) in the visible wavelength range. Therefore, the presence of blood in a cavity can obscure the view of a surgeon during surgery. Not all liquids have such a high level of absorption in this wavelength range. Water, for example, has very high transmission in the visible wavelength range. Therefore, the presence of a liquid such as water is unlikely to obscure the view of an object or structure located behind the liquid in the visible wavelength range unless the amount of liquid is very high.

It will be appreciated that the absorption/transmission of light by a liquid is wavelength dependent such that the level of absorption or transmission of the light by a liquid may vary in accordance with the wavelength of that light.

Concerning the example of red wine, which is almost opaque in the visible wavelength range, it will be appreciated that the absorption of light by the red wine is much lower in the infrared wavelength range. That is, the transmission of the light by red wine increases in the infrared wavelength range. Hence, a liquid which appears almost opaque in the visible wavelength range may in fact be translucent or even transparent at wavelengths outside this visible range. This is because the processes of reflectance, absorption and scattering are wavelength dependent and occur much less for the liquid (e.g. red wine) at these wavelengths than in the visible light wavelength range.

In the present disclosure, the short wavelength infrared region is defined as wavelengths in the range of approximately 780 to 2500 nm.

Returning to FIG. 5A of the present disclosure, the second panel illustrates an image of the same scene as the first panel of FIG. 5A (i.e. the glass 5000 and bottle 5002). However, in contrast to the first panel of FIG. 5A (which illustrated an image recorded for the visible wavelength range only) the second panel of FIG. 5A illustrates an image recorded for both the visible and short wavelength infrared wavelength range. At these wavelengths the liquid in glass 5000 and bottle 5002 transmits a much high percentage of the incoming light. Accordingly, the liquid appears translucent. That is, in this image (which includes short wavelength infrared wavelengths) it is possible to see through the liquid in glass 5000 and 5002 to observe the structure behind the liquid.

Finally, the third panel of FIG. 5A illustrates an example which is taken infrared wavelengths longer than the wavelengths used in the second panel of FIG. 5A (no visible light is used in this image). The glass 5000 and the bottle 5002 are still visible. However, at these wavelengths, a very high percentage of the incoming light is transmitted by the liquid (i.e. reflectance, scattering and absorption are very low). Therefore, the liquid appears almost transparent to the light. Accordingly, even though the liquid (red wine) is still present in the glass 5000 and bottle 5002, a very clear image of the structure behind the liquid can be obtained, even though the liquid is opaque at visible wavelength ranges.

Of course, while the example of FIGS. 5A and 5B have been described with reference to a number of specific liquids (water and red wine) it will be appreciated that the present disclosure is not particularly limited in this regard. Indeed, embodiments of the present disclosure may be used in order to acquire a acquire a wavelength and polarization state of light for imaging a structure at least partially obscured, in the visible wavelength range, by a liquid other than water or red wine. For example, the present disclosure may be applied to acquire a wavelength and polarization state of light which can be used to see through other liquids including, for example, bodily liquids such as blood as described with reference to the example of FIG. 2 of the present disclosure.

Indeed, while an example absorption spectra for a liquid (water or red wine) has been shown in FIG. 5B of the present disclosure, it will be appreciated that different liquids will have different absorption spectra. In an example such as the situation described with reference to FIG. 2 of the present disclosure (i.e. a surgical procedure) the wavelength dependence of the liquid in relation to reflectance, absorption and scattering may be very different to the example of FIG. 2 of the present disclosure. As such, according to embodiments of the disclosure, apparatus 3000 is configured to analyse the liquid in order to acquire a wavelength and polarization state of the light which can be used in order to see through the liquid. In other words, the aim of the analysis performed by apparatus 3000 is to determine the amount of reflectance, absorption and scattering of the light for different wavelengths and polarizations of light in order to acquire a wavelength and polarization state of the light which can be used in order to see through the liquid (being the specific liquid obscuring the target structure).

In addition, in some examples, certain additional information can be obtained from the absorption spectrum. That is, in the example of FIG. 2 of the present disclosure (i.e. a surgical procedure) the absorption spectrum of the patient's blood can be used in order to obtain some vital parameters of the patient. For example, the absorption spectrum of the patient's blood can be used in order to continuously monitor the oxygen level in the patient's blood.

In accordance with embodiments of the disclosure, measurement of the reflectance and absorption is performed using a wavelength tuneable light source (i.e. light source 3002) which is thus configured to generate light of a certain wavelength and polarization state, wherein the certain wavelength of light is light within a range of wavelengths outside a visible wavelength range. In some examples, the range of wavelengths outside the visible wavelength range may be short wavelength infrared wavelengths of light, for example (e.g. approximately 780 to 2500 nm). However, the present disclosure is not particularly limited in this regard. In other examples, the range of wavelengths may be any wavelength range outside the visible range (e.g. outside 380 to 700 nm).

By controlling the light source 3002 with processing circuitry 3010, apparatus 3000 can control the light source 3002 to generate, in a sequence, light of at least one predetermined wavelength and polarization state to illuminate the liquid (where the at least one predetermined wavelength is a wavelength within the range of wavelengths outside the visible range).

The light source 3002 of apparatus 3000 is configured to generate light of a certain wavelength and polarization state, wherein the certain wavelength of light is a wavelength of light within a range of wavelengths outside a visible wavelength range. The type of light source 3002 is not particularly limited in accordance with embodiments of the disclosure. That is, any light source 3002 can be used provided that the light source 3002 is able to generate light of a certain wavelength within a range of wavelengths outside a visible wavelength range. For example, the light source 3002 could be a collection of light emitting diodes, an incandescent light source, or the like. The wavelength of the light which is produced by the light source 3002 can thus be tuned or controlled to the certain wavelength by activating different light emitting diodes within the light source 3002 or by using different filters to filter the light which is produced by the light source 3002 to the certain wavelength as desired.

Likewise, a number of polarizing elements may be provided either internally or externally to light source 3002 which enable light source 3002 to produce light of a certain polarization under the control of the processing circuitry 3010.

The light source 3002 is thus controlled by apparatus 3000 to illuminate a target (i.e. a specific liquid) with light of different wavelengths and polarizations in sequence. In other words, in some examples, the light source 3002 can be used to sweep through a predefined range and combination of wavelengths and polarization states such that the liquid is illuminated with light of each of the predefined wavelengths and polarization states in turn. This enables apparatus 3000 to analyse the liquid in order to acquire a wavelength and polarization state of the light for which the reflectance, scattering and polarization properties of the liquid are minimum.

In some examples, the analysis of the liquid can be done in a separate step before the actual measurement. For example, in the situation described with reference to FIG. 2 of the present disclosure (a surgical operation) blood could be taken from the patient before the operation and analysed using apparatus 3000. This enables the reflectance, absorption and scattering properties of the liquid to be determined prior to the stage at which the liquid obscures the image.

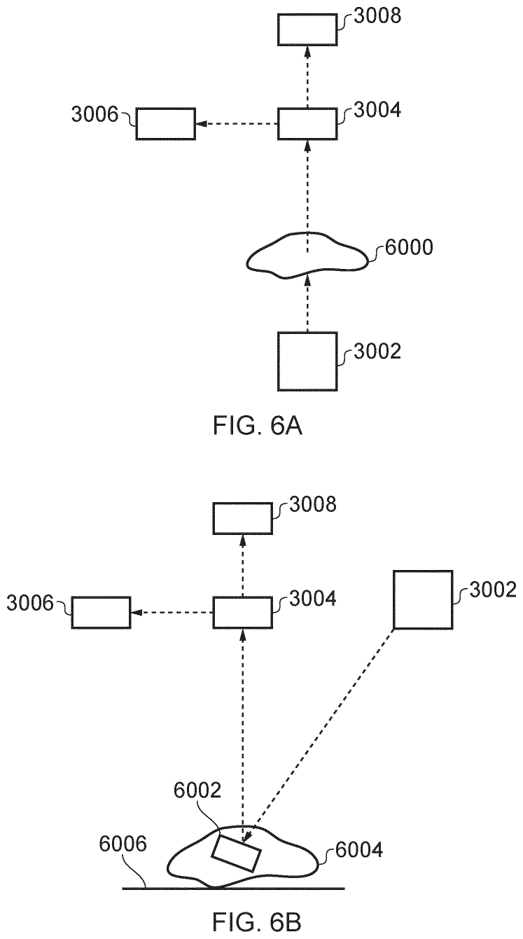

In such an example, the light source 3002 may be arranged on an opposite side of the liquid to the image capture device and the polarization detection unit, and wherein light from the liquid is light transmitted by the liquid from the light source. FIG. 6A illustrates an example configuration of the apparatus 3000 in accordance with embodiments of the disclosure.

In the example of FIG. 6A of the present disclosure, a sample of blood 6000 has been taken from a patient prior to a surgical operation. Apparatus 3000 is thus arranged in order to analyse the reflectance, absorption and scattering properties of the liquid from the sample of blood. Accordingly, the light source 3002 is arranged on an opposite side of the sample 6000 to the image capture device 3008 and the polarization detection unit 3006. This enables the light source to shine the light which has been generated through the sample, such that the image capture device 3008 and the polarization detection unit 3006 can be used in order to measure the reflectance, absorption and scattering properties of the liquid sample 6000.

This example configuration of apparatus 3000 enables the wavelength and polarization state of light which can be used to see through the liquid to be determined ahead of the surgical procedure.

However, in other examples, the analysis of the liquid can be done in a combined step with the actual measurement. For example, in the situation described with reference to FIG. 2 of the present disclosure (a surgical operation) blood, or indeed any other liquid 6004, could be analysed by the apparatus 3000 as it appears in the surgical scene 6006. In this situation, however, it is not necessarily possible to arrange the light source 3002 behind the liquid (i.e. on an opposite side of the liquid to the polarization detection unit 3006 and the image capture device 3008). Accordingly, a different configuration of apparatus 3000 may be required in this situation.

FIG. 6B illustrates an example configuration of the apparatus 3000 in accordance with embodiments of the disclosure. In this example, the light source 3002 is arranged on a same side of the liquid to the image capture device 3008 and polarization detection unit 3006. Additionally, a reflection unit 6002 is provided as part of apparatus 3000. Reflection unit 6002 is submersible in the liquid. Therefore, the reflection unit 6002 can be submersed in the liquid (e.g. blood) as it appears within the surgical environment.

In some examples, the reflection unit 6002 may be a mirror or other reflective tool submerged in the liquid 6004.

Light produced by the light source 3002 is then reflected from the reflection unit 6002 through the liquid (e.g. through the blood) towards the beam splitter (and thus towards the polarization detection unit 3006 and the image capture device 3008). Therefore, the light from the light source 3002 still passes through an amount of the liquid on route to the polarization detection unit 3006 and image capture device 3008. Hence, the reflectance, absorption and scattering properties of the liquid can be determined and used in order to acquire a wavelength and polarization state which can be used in order to see through the liquid.

This example configuration of apparatus 3000 enables the analysis of the liquid to be performed in situ, which means that apparatus 3000 can more efficiently adapt to changes in the liquid which occur during the surgical operation. Improved performance for imaging the target structure which is at least partially obscured by the liquid, in the visible wavelength, can thus be achieved.

### <Beam Splitting Element>

The beam splitting element 3004 of apparatus 3000 is configured to split light from a target, when the target is illuminated by the light source, along a first path and a second path.

In the example of FIGS. 6A and 6B of the present disclosure, the light from the target is light from the liquid.

By splitting the light from the target using the beam splitter 3004 along two paths, it is possible that both the polarization detection unit 3006 (arranged on the first path of the beam splitting element) and the image capture device (arranged on the second path of the beam splitting element) can be used concurrently to analyse the reflectance, absorption and scattering properties of the liquid. This improves the efficiency of apparatus 3000.

The form and configuration of the beam splitter is not particularly limited in accordance with embodiments of the disclosure. That is, any type of beam splitting device can be used in accordance with embodiments of the disclosure as desired.

### <Polarization Detection Unit 3006>

The polarization detection unit 3006 of apparatus 3000 is arranged on the first path of the beam splitting element and configured to measure a degree of polarization of light.

As explained with reference to FIG. 4 of the present disclosure, as light passes through the liquid some of that light will be scattered by the liquid. The scattering of light by the liquid is polarization dependent for each specific wavelength. When the level of scattering of the light is high, the liquid will appear translucent at best, even if the levels of reflectance and absorption are low. Therefore, in order to be able to see through the liquid and obtain a clear image of the structure, it is necessary to identify a polarization state of the light for which scattering by the liquid is minimum.

Accordingly, the polarization detection unit 3006 is used in order to measure a degree of polarization of the light. This can be used to determine the scattering of the light by the liquid at that specific wavelength.

In some examples, the polarization detection unit 3006 of apparatus 3000 may be a polarization camera which is configured to acquire polarization information (i.e. a degree of polarization of the light). However, in other examples, the polarization detection unit 3006 may be an image capture device provided with a number of configurable polarization elements. These configurable polarization elements may be used, with the image capture device, in order to acquire polarization information (i.e. a degree of polarization of the light). Accordingly, the polarization detection unit 3006 of apparatus 3000 is not particularly limited in accordance with embodiments of the disclosure, insofar that the polarization detection unit 3006 is able to measure a degree of polarization of the light.

In some examples, the degree of polarization of the light is an indication of the degree of depolarization. That is, the light which is produced by the light source 3002 is polarized light (having a certain polarization state). The polarization of this light will be weakened as it passes through the liquid owing to the scattering of the light. Hence, the degree of depolarization of the light (i.e. how much weaker the polarization of the light has become) provides a measure of the level of scattering of the light by the liquid. Indeed, in some examples, polarization unit 3006 may use a Muller Matrix to determine the scattering of light at a specific wavelength.

In some examples, the light source 3002 is configurable to generate light of at least 3 different polarization states for each specific wavelength in the wavelength range outside the visible wavelength range. The polarization detection unit 3006 can then be used in order to measure a degree of polarization of the light once it has travelled through the liquid for each of these 3 polarization states. The measure of the degree of polarization thus enables the level of scattering by the liquid for each of these 3 polarization states of light to be determined.

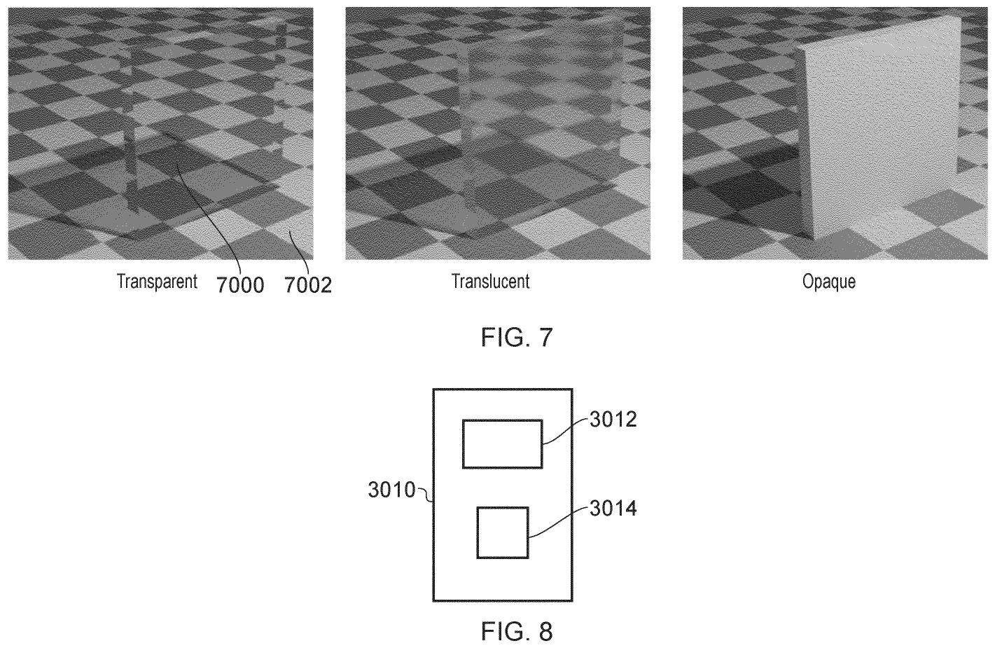

FIG. 7 illustrates an example of scattering of light in accordance with embodiments of the disclosure.

In this example, an object 7000 is shown in front of a structure 7002. In the first panel of FIG. 7, a first polarization state of the light is used. At this first polarization state, the level of scattering of light by object 7000 is very low. As such, the object 7000 appears transparent to the light at this first polarization state.

In the second panel of FIG. 7 of the disclosure, a second polarization state of light is used. At this second polarization state of the light, the level of scattering of light by object 7000 is increased. As such, the scattering of light means that object 7000 appears translucent to the light. Measuring the degree of polarization of the light once it has passed through the object 7000 at this second polarization state of light would show that the degree of polarization of the light was lower than at the first polarization state, thus indicating increased scattering of the light by the object.

Finally, in the third panel of FIG. 7 of the disclosure, a third polarization state of light is used in order to illuminate the object 7000. Here, the object 7000 appears opaque to the light owing to the very high level of scattering of light as it passes through the object 7000. Measurement of the degree of polarization of the light once it had passed through the object 7000 would show the very high level of scattering of the light.

As such, by controlling the light source 3002 to sequentially produce light of different polarization states and by measuring the degree of polarization (i.e. the strength of polarization) of the light once it has passed through the liquid using the polarization detection unit 3006, amount of scattering of light at different polarization states can be determined. This enables apparatus 3000 to identify a polarization state of light at which the liquid causes the minimum scattering of light.

Of course, it will be appreciated that while the example of FIG. 7 of the present disclosure has been described with reference to only 3 polarization states of light, the present disclosure is not actually limited in this regard. That is, any number of polarization states of the light can be used in the assessment of the level of scattering of the light by the liquid as required depending on the situation.

### <Image Capture Device>

The image capture device 3008 is arranged on the second path of the beam splitting element and configured to measure intensity of light at wavelengths within the range of wavelengths outside the visible wavelength range.

The wavelength range at which the image capture device is able to measure the intensity of light is not particularly limited in accordance with the present disclosure, as long as the image capture device is able to measure intensity of light outside the visible wavelength range (e.g. 380 to 700 nm, for example). In some examples, the image capture device may be a camera configured to detect light in short wavelength infrared or near-infrared wavelengths (e.g. approximately 780 to 2500 nm). This enables the image capture device to measure intensity of light at wavelength ranges outside the visible wavelength range in a range at which the liquid is most likely to have minimum reflection, absorption and scattering properties.

Indeed, in some examples, the image capture device 3008 of apparatus 3000 is configured to detect light in the short wavelength infrared or near-infrared wavelength range of light generated by the light source 3002.

The type of image capture device used in accordance with embodiments of the present disclosure is not particularly limited. In some examples, the image capture device 3008 may be configured as a camera. In other examples, the image capture device may comprise a photo-diode or the like. Indeed, any type of image capture device can be used provided that the image capture device measures the intensity of the light in the wavelength range outside the visible wavelength range.

In some examples, a colour filter may be provided between the liquid and the beam splitting element 3004 and/or between the beam splitting element and the image capture device 3008. The colour filter may be configured in order to block the visible wavelength range. This ensures that the light from the visible wavelength range does not interfere with the image capture device configured to measure the intensity of the light in the wavelength range outside the visible wavelength range.

In some examples, the image capture device may capture an image of the scene when measuring the intensity of light at the wavelength range outside the visible wavelength range. The image of the scene captured by the image capture device may further be displayed on an external display device such that the image of the scene can be viewed by a user.

### <Processing Circuitry>

FIG. 8 illustrates an example configuration of an apparatus in accordance with embodiments of the disclosure. That is, an example configuration of the circuitry 3010 of apparatus 3000 is shown in FIG. 8 of the present disclosure. The circuitry 3010 (e.g. processing circuitry) is configured as a control unit 3012 configured to control the light source 3002 to generate, in a sequence, light of at least one predetermined wavelength and polarization state to illuminate a liquid. Then, for light of each of the at least one predetermined wavelength and polarization state, the control unit 3012 is configured to determine a level of scattering of the light by the liquid based on the degree of polarization of light measured by the polarization detection unit 3006 and determine a level of reflectance and absorption of the light by the liquid based on the intensity of light measured by the image capture device 3008.

Consider the example of FIG. 6A of the present disclosure (where the apparatus 3000 is used on a sample of the liquid). The control unit 3012 of circuitry 3010 of apparatus 3000 controls the light source 3002 such that the light source generates light at different wavelengths in a sequence. For example, the light source can be controlled to generate light (illumination) at fixed steps across the wavelength range of the light source (e.g. at fixed steps between 400 nm and 1700 nm in the case of the light source capable of generating light at SWIR wavelengths). However, fixed steps between these wavelengths need be used by the control unit 3012. Rather, any sequence of different wavelengths of light may be used as required. Moreover, different polarization states of this light can also be produced by the light source in sequence under the control of circuitry 3010.

In this manner, liquid 6000 is illuminated with light of the different wavelengths and polarization states in a sequence.

For each of these different wavelengths and polarization states, the polarization detection unit 3006 and the image capture device 3008 can measure the degree of polarization of the light (i.e. how strongly the light of is polarized) and the intensity of the light once it has passed through the liquid 6000. Accordingly, as discussed with reference to FIG. 4 of the present disclosure, the circuitry is able to determine a level of scattering of the light (based on the degree of polarization of the light) and a level of reflection and absorption of the light (based on the intensity of the light) by the liquid 6000 for each of these different wavelengths and polarization states.

In this manner, the apparatus 3000 can analyse the reflectance, absorption and scattering properties of the liquid 6000 at the different wavelength and polarization states of light.

Then, acquiring unit 3014 of circuitry 3010 of apparatus 3000 is configured to acquire a wavelength and polarization state of light for imaging a structure at least partially obscured, in the visible wavelength range, by the liquid in accordance with the level of scattering, reflectance and absorption of light which has been determined for each of the at least one predetermined wavelength and polarization state.

Consider, again, the example of FIG. 6A of the present disclosure. In this example, acquiring unit 3014 acquires the best wavelength and polarization state of the light for imaging the structure by identifying the combination of wavelength and polarization state of light at which the liquid appears the most transparent. In other words, the acquiring unit 3014 identifies the wavelength and polarization state of light which can be used in order to see through the liquid.

In some examples, the acquiring unit 3014 may acquire the wavelength and polarization state of light by finding the wavelength and polarization state of light from amongst the predetermined wavelength and polarization state which provides the minimum scattering, reflectance and absorption of light by the liquid.

However, in other examples, the acquiring unit 3014 may first acquire the wavelength of light for which the levels of reflectance and absorption of the light are minimum. This can be identified on the measured intensity of light from the image capture device at each of the wavelengths. Then, once the wavelength of light has been acquired, the acquiring unit 3014 may acquire the polarization state of the light which provides the minimum amount of scattering of light.

More specifically, in some examples, minima in the absorption spectrum (constructed from the intensity of light measured by the image capture device) can be used to identify potential “see-through candidates”. These are wavelengths of light for which the level of absorption of light by the liquid is very low. Then, minima in a spectral reflectance curve (reflectance against wavelength) can be used in order to identify which of those “see-through” candidates (having low levels of absorption) also have very low levels of reflectance. The “see-through” candidate with the lowest combined levels of absorption and reflectance can then be chosen as the best candidate for a wavelength of light which can “see through” the liquid. Finally, the different polarization states of the light at that wavelength can then be analysed to determine the polarization at the given wavelength which results in the liquid having the lowest level of scattering of light. The combination of wavelength and polarization state can then be acquired by the acquiring unit as the best wavelength and polarization state of the light for imaging a structure at least partially obscured by the liquid (as this is the combination of wavelength and polarization state at which the liquid is most transparent to the light).

Of course, it will be appreciated that the present disclosure is not particularly limited to these specific examples, and that the circuitry 3010 of apparatus 3000 may acquire the wavelength and polarization state of light for imaging a structure at least partially obscured by the liquid in any way using the level of reflectance, scattering and absorption of the light as required depending on the situation.

### <Advantageous Technical Effects>

Accordingly, apparatus 3000 is configured to acquire a wavelength and polarization state of light for imaging a structure at least partially obscured, in the visible wavelength range, by a liquid. Light of this acquired wavelength and polarization state can be used in order to see through the liquid thus providing an image with a better view of the structure.

### <Method>

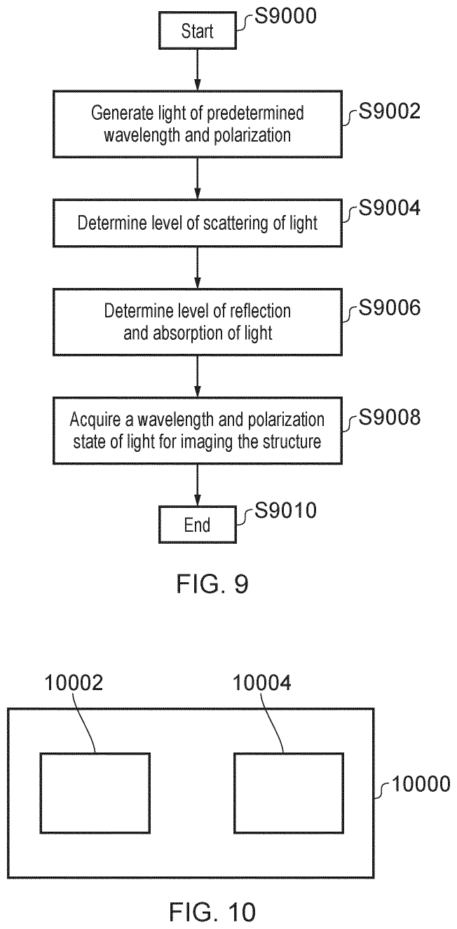

FIG. 9 illustrates a method in accordance with embodiments of the present disclosure. The method illustrated in FIG. 9 of the present disclosure may be implemented by an apparatus such as apparatus 1000 for controlling an apparatus such as apparatus 3000 of the present disclosure, for example.

The method starts at step S9000 and proceeds to S9002.

In step S9002, the method comprises controlling a light source 3002 to generate, in a sequence, light of at least one predetermined wavelength and polarization state to illuminate a liquid.

Then, in step S9004, for light of each of the at least one predetermined wavelength and polarization state, the method comprises determining a level of scattering of the light by the liquid based on the degree of polarization of light measured by a polarization detection unit 3006.

In step S9006, the method comprises determining a level of reflectance and absorption of the light by the liquid based on the intensity of light measured by the image capture device 3008.

Then, in step S9010, the method comprises acquiring a wavelength and polarization state of light for imaging a structure at least partially obscured, in the visible wavelength range, by the liquid in accordance with the level of scattering, reflectance and absorption of light which has been determined for each of the at least one predetermined wavelength and polarization states.

The method proceeds to, and ends with, step S9010.

### <Apparatus>

As explained with reference to FIG. 3 of the present disclosure, apparatus 3000 is configured in order to acquire a wavelength and polarization state of light which can be used in order to image a structure which is at least partially obscured by a liquid in the visible wavelength range. This is achieved by analysing the liquid and acquiring a wavelength and polarization state of light at which the transparency of the liquid is highest (based on the levels of reflectance, absorption and scattering of the light caused by the liquid). Accordingly, once apparatus 3000 has acquired the wavelength and polarization state of the light in this manner, these can be used in order to see through the liquid and image a structure at least partially obscured by the liquid.

FIG. 10 illustrates an example configuration of an apparatus in accordance with embodiments of the disclosure. The apparatus 10000 is an apparatus for imaging a structure at least partially obscured, in a visible wavelength range, by a liquid. The apparatus 10000 comprises a light source 10002 configured to illuminate the structure with light of a predetermined wavelength and polarization state, the predetermined wavelength and polarization state being acquired, for example, by apparatus such as apparatus 3000 of the present disclosure.

The light source 10002 of apparatus 10000 may, in some examples, be the same as the light source 3002 of apparatus 3000. Indeed, in some examples, the light source may be able to generate polarized short wavelength infrared light. However, the present disclosure is not particularly limited in this regard, provided that the light source is able to generate light of the wavelength and polarization state acquired by apparatus 3000.

Apparatus 10000 further comprises a second image capture device 10004 configured to generate an image of the structure, as illuminated by the light source, at the predetermined wavelength and polarization state. The second image capture device 10004 of apparatus 10000 may, in some examples, by the same as the image capture device 3008 described with reference to FIG. 3 of the present disclosure. Indeed, in some examples, the second image capture device 10004 may be a short wavelength infrared camera. However, the present disclosure is not particularly limited in this regard, provided that the second image capture device 10004 is able to capture an image of the tissue at the wavelength generated by the light source 10002.

In this manner, apparatus 10000 is able to use the predetermined wavelength and polarization state of light in order to see through the liquid which at least partially obscures a structure in the visible wavelength range and thus provide an image with a better view of the structure. This enables more detailed information of the scene to be obtained.

### <Example Situation>

Consider again the example situation explained with reference to FIG. 2 of the present disclosure. Here, a surgeon 2002 is performing a surgical operation on a patient 2004. An image captured by an image capture device (not shown) is displayed to the surgeon 2002 on a display device 2010. Accordingly, the surgeon 2002 can view images of the surgical scene (i.e. a part of the patient currently being operated upon) on the display device 2010. This assists the surgeon 2002 in their performance of the surgical operation.

As the surgical procedure progresses, a significant part of the tissue of the patient may become covered by blood and/or other liquids. Indeed, the human body consists of approximately 70% water. Therefore, during surgery, it is very likely that the image from the image capture device may be obscured by some body liquids of the patient (such as blood, for example). As such, during the surgery, it may become very difficult for the surgeon 2002 to be able to see the target tissue (i.e. the part of the patient currently being operated on) in the image from displayed on display device 2010.

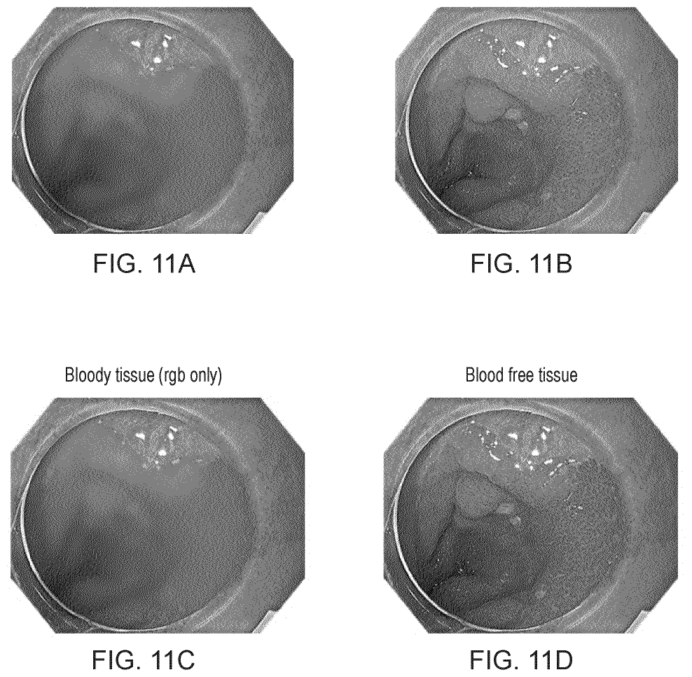

FIG. 11A of the present disclosure illustrates an example image in accordance with embodiments of the disclosure. This image is an image which may be displayed to the surgeon on display device 2010 during the surgery, as blood (or other liquids) begin to obscure the tissue.

However, in accordance with embodiments of the disclosure, apparatus 3000 may acquire a wavelength and polarization state of light which can be used in order to see through the liquid and enable the surgeon to see a clear image of the underlying tissue. As explained with reference to FIGS. 6A and 6B of the present disclosure, the liquid can be analysed either by taking a sample of the liquid prior to the surgery or in situ during the surgical procedure itself (e.g. as the blood appears in the image). Once apparatus 3000 has acquired the wavelength and polarization state of the light, apparatus 10000 can use this wavelength and polarization state of the light in order to see through the liquid and provide the surgeon with a clear image of the underlying tissue.

That is, light source 10002 of apparatus 10000 can illuminate the surgical scene with light of the wavelength and polarization state acquired by apparatus 3000. Since the liquid has very low levels of reflectance, absorption and scattering for this illumination, it will appear transparent to this light. As such, the second image capture device 10004 can acquire an image of the tissue at this wavelength. This image of the tissue can then be provided to the surgeon on the display screen 2010 such that the surgeon 2002 can have a clear view of the tissue beneath the blood.

FIG. 11B illustrates an example image in accordance with embodiments of the disclosure Specifically, FIG. 11B illustrates an image of the tissue taken through the liquid (i.e. at the wavelength and polarization state acquired by apparatus 3000) is shown in FIG. 11B of the present disclosure. With an image such as the example image of FIG. 11B, a clear view of the tissue can be obtained, even though the tissue is obscured, at least partially, in the visible wavelength range by the liquid. In this example, FIG. 11B is a monochrome image of the scene because the image of FIG. 11B is taken at a single wavelength corresponding to the illumination generated by the light source 10002.

### <Colour Reconstruction>

While the second image capture device 10004 can be used in order to capture an image of a structure through a liquid (as explained with reference to FIGS. 11A and 11B of the present disclosure) it will be appreciated that the image captured by the second image capture device 10004 may be an image at a single wavelength. The see-through image generated by the second image capture device 10004 may be a monochrome image of the structure (as it is an image captured at the wavelength which has been acquired by apparatus 3000). Accordingly, colour information regarding the structure under the liquid may be lost.

As such, in some embodiments of the disclosure, the apparatus 10000 may further comprise a third image capture device configured to generate a third image of the structure, as illuminated by the light source, at the visible wavelength range; and circuitry configured to combine the second image from the second image capture device and the third image from the third image capture device to create an output image of the structure.

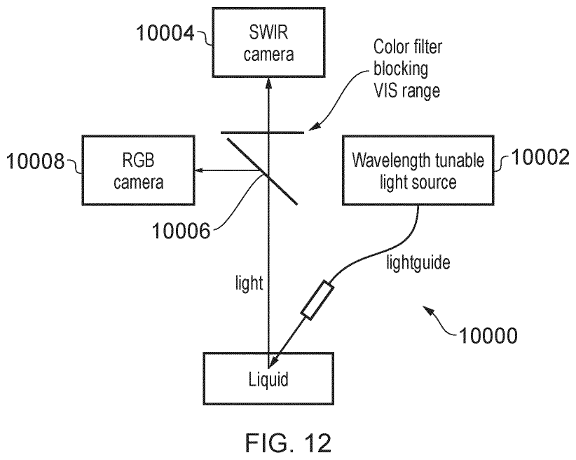

Consider now FIG. 12 of the present disclosure. FIG. 12 illustrates an apparatus according to embodiments of the disclosure.

The apparatus 10000 illustrated in FIG. 12 of the present disclosure comprises a light source 10002 (wavelength tuneable light source) a second image capture device 10004 (SWIR camera) a beam splitter 10006 and a third image capture device 10008 (RGB camera).

Apparatus 10000 can be used in order to obtain an image of a structure at least partially obscured in the visible wavelength range. That is, the light source 10002 generates light of a predetermined wavelength and polarization state which illuminates the liquid. However, the liquid is transparent to the light of this predetermined wavelength and polarization state. Accordingly, the light passes through the liquid to the structure beneath. Then, the light reflects from the surface of the structure beneath the liquid and passes back through the liquid towards the beam splitter. The beam splitter splits this light onto two paths. The first path passes towards the second image capture device 10004 of apparatus 10000. This image capture device is able to capture an image of the tissue at the wavelength generated by the light source 10002. Accordingly, image capture device 10004 sees through the liquid and captures an image of the structure beneath the liquid.

Additionally, light source 10002 generates light within the visible wavelength range (i.e. in addition to the light of the predetermined wavelength and polarization state). This light in the visible wavelength range illuminates the liquid. Light source 10002 can generate the light within the visible range and the light of the acquired wavelength and polarization at the same time. However, in contrast to the light of the predetermined wavelength and polarization state, the liquid is not transparent to the light at in the visible wavelength range. Accordingly, the visible light does not travel through the liquid to the surface of the structure beneath the liquid. However, light (in the visible wavelength range) reflects from the structure which is not covered by the liquid and light (in the visible wavelength range) reflects from the surface of the liquid towards the beam splitter. The beam splitter splits this light onto two paths. The first path passes towards the second image capture device 10004. However, a colour filter blocking the visible wavelength range may be provided in order to prevent light of the visible wavelength range reaching the second image capture device (and thus from interfering with the image captured by the second image capture device). The second path of the beam splitter passes towards the third image capture device 10008. This image capture device is an image capture device capable of capturing an image in the visible wavelength range. Accordingly, the third image capture device 10008 is able to capture a visible wavelength image of the scene. This may, for example, be an RGB image of the scene, for example.

As such, even though the third image capture device 10008 is not able to see through the liquid (as the liquid at least partially obscures the structure in the visible wavelength range) the third image capture device 10008 is able to capture a colour image of the scene in the visible wavelength range (e.g. an RGB image or the like).

Accordingly, apparatus 10000 may be configured to combine the second image from the second image capture device 10004 (e.g. the see-through image) and the third image from the third image capture device 10008 (e.g. the RGB image) to create an output image of the structure. This enables the apparatus 10000 to reconstruct the original tissue colour behind the liquid (e.g. by blending the second and third image).

FIG. 11C illustrates an example image in accordance with embodiments of the disclosure. This image is an image which may be displayed to the surgeon on display device 2010 during the surgery, as blood (or other liquids) begin to obscure the tissue. As the liquids begin to obscure the image, the surgeon may no longer be able to view the tissue.

FIG. 11D illustrates an example image in accordance with embodiments of the disclosure. The image of FIG. 11D is an image which is captured at the same time during surgery as FIG. 11C of the present disclosure (i.e. as blood, or other liquids, begin to obscure the tissue). This image is an image which may be captured by apparatus 10000 as described with reference to FIG. 12 of the present disclosure. Using the second image capture device 10004, apparatus 10000 is able to see through the liquid to the underlying structure. Moreover, using the third image capture device, apparatus 10000 is able to obtain an RGB image of the scene. By combining these images, apparatus 10000 is able to reconstruct the original tissue colour behind the blood in a final output image (the image shown in FIG. 11D). This final output image may be displayed on a display device (such as display device 2010) such that an improved image of the structure behind the liquid can be observed.

The manner by which apparatus 10000 combines the second and third image in order to perform colour reconstruction is not particularly limited in accordance with embodiments of the disclosure.

However, in a first example, the see-through image (from the second image capture device 10004) can be taken as a “base” image and the RGB image (from the third image capture device 10008) can be used as an additional input to a deep-learning architecture. The deep-learning architecture may then perform colourization of the whole “see through” image based on the RGB image. In other words, the deep-learning model performs colourization of the monochrome see-through image with the RGB image (from the third image capture device) as a reference. The deep-leaning architecture may have be trained on a set of training data. Indeed, any trained model may be used in order to perform colourization of the image from the second image capture device 10004 with the image from the third image capture device 10008 as required.

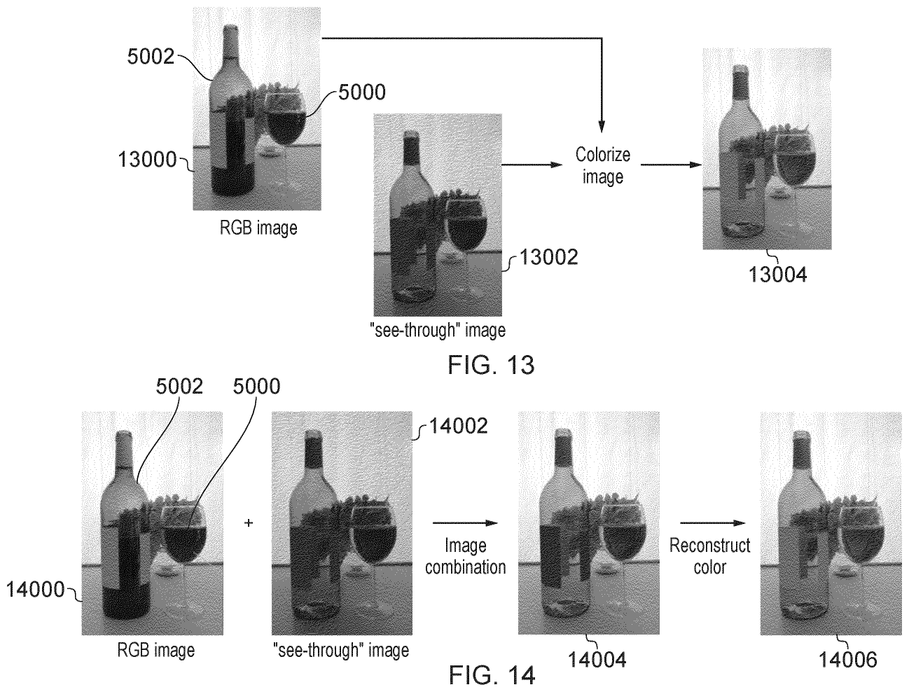

FIG. 13 of the present disclosure illustrates an example colourization process in accordance with embodiments of the disclosure. In this example a glass 5000 and bottle 5002 are shown. A liquid (red wine) is contained within each of the glass 5000 and bottle 5002. This liquid has high levels of reflectance, absorption and scattering at visible wavelengths (e.g. 380 to 700 nm). As such, the liquid appears opaque at visible wavelengths.

In this example, image 13000 is an RGB image (captured by the third image capture device of apparatus 10000). This image is a colour image of the scene. However, since it an image which has been obtained at visible wavelengths, the liquid appears opaque such that the liquid obscures the view of any object behind the liquid.

In contrast, image 13002 is a see through image of the scene (captured by the second image capture device of apparatus 10000). This is a monochrome image of the scene, since it has been taken for light of a certain wavelength and polarization (being the wavelength and polarization state of light acquired by apparatus 3000). However, since the liquid (red wine) is transparent for this wavelength and polarization of light (i.e. has low reflectance, absorption and scattering of the light) then it is possible to see through the liquid in order to observe features or structures behind the liquid.

Finally, image 13004 is an output image generated by combining image 13002 and image 13004. That is, a trained model (such as a deep learning model) is used in order to perform colourization of the whole “see through” image based on the RGB image. The combined image 13004 enables a colour image of the structure obscured behind the liquid to be obtained.

Of course, it will be appreciated that the present disclosure is not particularly limited to this specific example illustrated in FIG. 13 of the present disclosure.

Alternatively, in a second example, circuitry of apparatus 10000 may be configured to first analyse the images from the second and third image capture devices in order to determine which portions of the images correspond to liquid (i.e. to identify where in the image the liquid is located). Only the portions of the image with liquid will require the use of the see through image (from the second image capture device). Classification of each pixel of the image as liquid or non-liquid can be performed in a number of different ways and is not particularly limited in accordance with the present disclosure. Indeed, in some examples, the classification task can be performed by machine learning algorithms (such as support vector machine or random forest classifier machine learning algorithms). However, the classification task can also be performed by a deep learning process depending on the situation.

Once the classification of pixels as liquid and non-liquid has been performed, apparatus 10000 can select the corresponding pixels of the third image capture device (e.g. the RGB image) as the pixels of the output image for pixels which have been classified as non-liquid. However, the pixels from the see through image (the image from the second image capture device) are required to be used when the pixels are classified as liquid (in order that the structure beneath the liquid can be observed). As explained above, the pixels from the see through image are greyscale and must be coloured. The colourization of these pixels can be performed by a trained model such as a deep learning model. However, in contrast to the first example, the colourization of the pixels is applied only to the pixels which have been classified as liquid (since the non-liquid pixels are taken directly from the RGB image).

FIG. 14 of the present disclosure illustrates an example colourization process in accordance with embodiments of the disclosure. The example of FIG. 14 demonstrates an example of colourizing the image which has been captured by the second image capture device 10004 by classifying the pixels of the image as liquid or non-liquid. In this example a glass 5000 and bottle 5002 are shown. A liquid (red wine) is contained within each of the glass 5000 and bottle 5002. This liquid has high levels of reflectance, absorption and scattering at visible wavelengths (e.g. 380 to 700 nm). As such, the liquid appears opaque at visible wavelengths.

In this example, image 14000 is an RGB image (captured by the third image capture device of apparatus 10000). This image is a colour image of the scene. However, since it an image which has been obtained at visible wavelengths, the liquid appears opaque such that the liquid obscures the view of any object behind the liquid.

In contrast, image 14002 is a see through image of the scene (captured by the second image capture device of apparatus 10000). This is a monochrome image of the scene, since it has been taken for light of a certain wavelength and polarization (being the wavelength and polarization state of light acquired by apparatus 3000). However, since the liquid (red wine) is transparent for this wavelength and polarization of light (i.e. has low reflectance, absorption and scattering of the light) then it is possible to see through the liquid in order to observe features or structures behind the liquid.

In this example, the pixels of the images 14000 and 14002 are first classified as liquid or non-liquid (using a trained model). Then, for the pixels classified as liquid, the corresponding pixels from the see through image 14002 are used as the pixels of the output image. In contrast, when a pixel is classified as non-liquid, the corresponding pixel from the RGB image is used as the pixel of the output image. Thus, a combined image 14004 is created using pixels from the RGB image 14000 and pixels from the see through image 14002 depending on whether those pixels are classified as liquid or non-liquid.

Finally, once the images have been combined, apparatus 10000 performs a colour reconstruction process on the image. That is, the pixels taken from the see through image 14002 are still grayscale and must be coloured in a separate step. A trained model (such as a deep learning model) can be used in order to perform this colourization in certain examples of the present disclosure. The colourized combined image 14006 is used an output image. This process enables a colour image of the structure obscured behind the liquid to be obtained.

Of course, it will be appreciated that the present disclosure is not particularly limited to this specific example illustrated in FIG. 14 of the present disclosure. Indeed, any suitable process for combing the images from the second and third image capture devices can be used in accordance with the present disclosure as required depending on the situation to which embodiments of the disclosure are applied.

### <Additional Modifications>

As explained with reference to FIG. 10 of the present disclosure, apparatus 10000 is able to use the wavelength and polarization state of light (determined by apparatus 3000) in order to generate an image of a structure which is at least partially covered or obscured by a liquid in a visible wavelength range.

However, it will be appreciated that the apparent transparency of a liquid may depend also upon the thickness of that liquid. That is, a thicker layer of liquid will more strongly attenuate the light as it passes through the liquid than a thinner layer of the same liquid. This is because the light has to pass through more liquid. Accordingly, the amount of scattering and absorption of the light, for example, will be higher when the thickness of the liquid increases.

Therefore, if the thickness of the liquid increases, the see through image obtained of the structure beneath the liquid may become less clear (since more of the light from light source 10002 will be attenuated by the liquid). In some examples, when the level of the liquid increases, it may be advantageous to increase the intensity of the light source 10002, such that even if the attenuation of the light from the liquid increases, an image of the structure beneath the liquid can still be obtained.

As such, in some examples of the present disclosure, the circuitry is configured to control the intensity of the light source in accordance with an amount of liquid which obscures the structure, and wherein the amount of liquid obscuring the structure is determined in accordance with at least one of the image captured by the second and third image capture device, a signal from an external device and/or an input from a user.

The control of the intensity of the light source may include, for example, increasing the intensity of the light source when the amount of liquid increases.

The method of determining when the level of the liquid increases is not particularly limited in accordance with embodiments of the disclosure. In some examples, the level of the liquid may be determined in accordance with the appearance of the image captured by the second image capture device (e.g. the see through image) or the appearance of the image captured by the third image capture device (e.g. the RGB image). Alternatively, an external device (such as a different tool being used) may be used to identify when the level of the liquid increases. Further alternatively, the user may provide an input requesting that the intensity of the light is increased when the level of the liquid increases.

In this manner, the intensity of the light from light source 10002 can be increased in order that an image of the structure beneath the liquid can be obtained even when the level of liquid present in the scene increases.

### <Method>

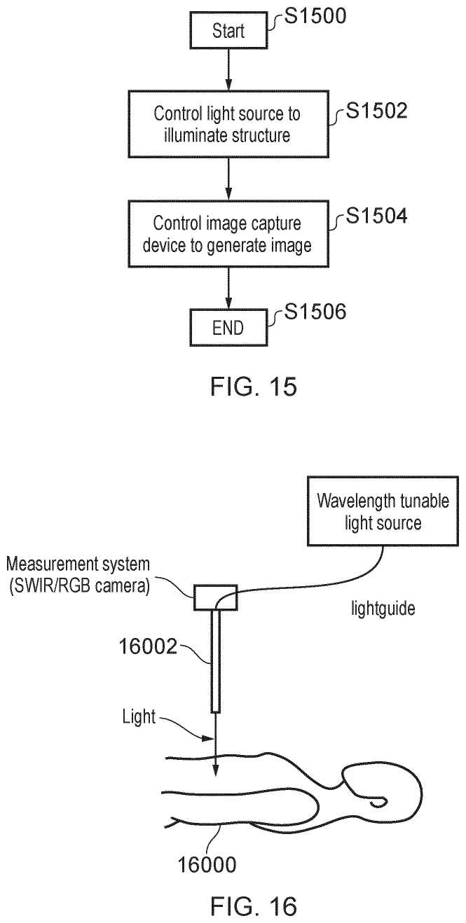

FIG. 15 illustrates a method in accordance with embodiments of the present disclosure. The method illustrated in FIG. 15 of the present disclosure may be implemented by an apparatus such as apparatus 1000 for controlling an apparatus such as apparatus 10000 of the present disclosure, for example.

The method starts at step S1500 and proceeds to S1502.

In step S1502, the method comprises controlling a light source to illuminate a structure with light of a predetermined wavelength and polarization state, the predetermined wavelength and polarization state. The predetermined wavelength and polarization state may be the wavelength and polarization state acquired by apparatus 3000, for example.

Then, in step S1504, the method comprises controlling an image capture device to generate a second image of the structure, as illuminated by the light source, at the predetermined wavelength and polarization state.

The method then proceeds to, and ends with, step S1506.

### Implementation Examples

In some examples, apparatus 3000 and/or apparatus 10000 may be implemented as part of an endoscopic device.

FIG. 16 illustrates an example endoscope system in accordance with embodiments of the disclosure.

In this example, endoscopic surgery is being performed on a patient 16000 using a surgical endoscope 16002. Image capture devices of apparatus 3000 and/or apparatus 10000 (such as a SWIR/RGB camera or the like) may be included at the end of the endoscope 16002. The light source of apparatus 3000 and/or apparatus 10000 (i.e. a wavelength tuneable light source) can be attached by means of a light guide to the surgical endoscope such that light from the light guide illuminates the surgical scene. This enables efficient use of apparatus 3000 and/or apparatus 10000 during a surgical endoscope procedure.

Furthermore, in some examples, at least a portion of apparatus 3000 and/or apparatus 10000 may be incorporated into other devices such as surgical tools or the like. For example, a reflective unit (e.g. a reflective mirror or the like) can be included on one side of an instrument (such as surgical scissors or the like). Light reflected from the surface of this reflective unit can then be used by apparatus 3000 in order to analyse the liquid once the reflective unit is submerged in the liquid.

Alternatively, in some examples, apparatus 3000 and/or apparatus 10000 may be implemented as a chip-on-tip solution which has direct contact with the blood or other liquid to be removed from the image.

Of course, it will be appreciated that the present disclosure is not particularly limited to these specific examples. The apparatuses of the present disclosure can be implemented in a number of different systems, tools and devices depending on the context of the situation to which the embodiments of the disclosure are applied.

In fact, while certain embodiments of the present disclosure have been described with reference to an example situation where an imaging device is used during surgery, it will be appreciated that the present disclosure is not particularly limited in this regard. For example, embodiments of the disclosure can be applied to video endoscopy. In the case of video endoscopy, it is very difficult to manually remove liquids from an image (since it can be very difficult to use tools, such as a suction device, to remove the liquid). Therefore, the ability to image the target while seeing through the liquid in accordance with embodiments of the disclosure is very advantageous. Moreover, the embodiments of the disclosure are not limited to the removal of blood from an image (i.e. seeing through the blood). Different liquids in different parts of the body can be analysed in order to identify a wavelength and polarization state at which those liquids appear the most transparent. These wavelengths and polarization states of the light can then be used in order to see through these different liquids as required.

Indeed, embodiments of the disclosure can also be applied to many other example situations (including example situations outside the surgical or medical environment). In particular, embodiments of the present disclosure may be applied to certain engineering situations and/or industrial environments. In particular, embodiments of the disclosure may be used in any situation where an object or structure to be imaged is at least partially obscured by a liquid in the visible wavelength range. Hence, embodiments of the disclosure may also be applied to an example situation such as observation of a partially submerged structure (such as a pipe).

Alternatively, embodiments of the disclosure may also be used in biological settings, to improve images which can be obtained of an aquatic species, for example. As such, the present disclosure is not particularly limited in this regard.

In addition, technical features and aspects of the present disclosure may further be arranged in accordance with the following numbered clauses:

1) An apparatus for acquiring a wavelength and polarization state of light for imaging a structure at least partially obscured, in a visible wavelength range, by a liquid, the apparatus comprising:
a light source configured to generate light of a certain wavelength and polarization state, wherein the certain wavelength of light is a wavelength of light within a range of wavelengths outside a visible wavelength range;
a beam splitting element to split light from a target, when the target is illuminated by the light source, along a first path and a second path;
a polarization detection unit arranged on the first path of the beam splitting element and configured to measure a degree of polarization of light;
an image capture device arranged on the second path of the beam splitting element and configured to measure intensity of light at wavelengths within the range of wavelengths outside the visible wavelength range;
and circuitry configured to:
control the light source to generate, in a sequence, light of at least one predetermined wavelength and polarization state to illuminate a liquid;
and, for light of each of the at least one predetermined wavelength and polarization state, determine a level of scattering of the light by the liquid based on the degree of polarization of light measured by the polarization detection unit;
determine a level of reflectance and absorption of the light by the liquid based on the intensity of light measured by the image capture device;
and wherein the circuitry is further configured to acquire a wavelength and polarization state of light for imaging a structure at least partially obscured, in the visible wavelength range, by the liquid in accordance with the level of scattering, reflectance and absorption of light which has been determined for each of the at least one predetermined wavelength and polarization state.

2) The apparatus according to clause 1, wherein the image capture device is configured to measure intensity of light at wavelengths in the range of 400 nm to 1700 nm.

3) The apparatus according to clause 1 or 2, wherein the light source comprises a plurality of light emitting diodes and/or colour filters for generating the sequence of light of at least one predetermined wavelength and polarization state.

4) The apparatus according to any preceding clause, wherein the circuitry is configured to acquire the wavelength and polarization state of light by determining the wavelength and polarization state of light from amongst the predetermined wavelength and polarization state which provides the minimum scattering, reflectance and absorption of light by the liquid.

5) The apparatus according to an preceding clause, wherein the light source is arranged on an opposite side of the liquid to the image capture device and the polarization detection unit, and wherein light from the liquid is light transmitted by the liquid from the light source.

6) The apparatus according to any preceding clause, wherein the light source is arranged on a same side of the liquid to the image capture device and polarization detection unit; wherein the apparatus further comprises a reflection unit submersible in the liquid; and wherein light from the liquid is light reflected from the reflection unit through the liquid from the light source.

7) An apparatus for imaging a structure at least partially obscured, in a visible wavelength range, by a liquid, the apparatus comprising:
a light source configured to illuminate the structure with light of a predetermined wavelength and polarization state, the predetermined wavelength and polarization state being acquired by an apparatus according to any of clauses 1 to 6; and
a second image capture device configured to generate a second image of the structure, as illuminated by the light source, at the predetermined wavelength.

8) The apparatus according to clause 7, wherein the light source is further configured to generate light of the visible wavelength region; and wherein the apparatus further comprises:
a third image capture device configured to generate a third image of the structure, as illuminated by the light source, at the visible wavelength range;
and circuitry configured to combine the second image from the second image capture device and the third image from the third image capture device to create an output image of the structure.

9) The apparatus according to clause 8, wherein the circuitry is configured to combine the second image from the second image capture device and the third image from the third image capture device by colouring pixels of the second image based on the pixels of the third image.

10) The apparatus according to clause 8, wherein the circuitry is configured to combine the second image from the second image capture device and the third image from the third image capture device by:
classifying each pixel of the image from each of the second and third image capture device as liquid or non-liquid;
using the pixel from the third image as the corresponding pixel of the output image when the pixel is non-liquid and using the pixel from the second image as the corresponding pixel of the output image when the pixel is classified as liquid;
and colouring the pixels of the output image corresponding to the second image based on the pixels of the third image.

11) The apparatus according to clause 9 or 10, wherein the circuitry is configured to perform the classifying and/or colouring using a machine learning and/or deep learning process.

12) The apparatus according to any of clauses 8 to 11, wherein the circuitry is configured to control the intensity of the light source in accordance with an amount of liquid which obscures the structure, and wherein the amount of liquid obscuring the structure is determined in accordance with at least one of the image captured by the second and third image capture device, a signal from an external device and/or an input from a user.

13) An endoscopic device comprising the apparatus according to clauses 1 to 6 and/or the apparatus according to clauses 7 to 12.

14) A method of acquiring a wavelength and polarization state of light for imaging a structure at least partially obscured, in a visible wavelength range, by a liquid, the method comprising:
generating, using a light source, light of a certain wavelength and polarization state, wherein the certain wavelength of light is a wavelength of light within a range of wavelengths outside a visible wavelength range;
splitting, using a beam splitting element, light from a target, when the target is illuminated by the light source, along a first path and a second path;
measuring a degree of polarization of light using a polarization detection unit arranged on the first path of the beam splitting element;
measuring intensity of light at wavelengths within the range of wavelengths outside the visible wavelength range using an image capture device arranged on the second path of the beam splitting element;
controlling the light source to generate, in a sequence, light of at least one predetermined wavelength and polarization state to illuminate a liquid;
and, for light of each of the at least one predetermined wavelength and polarization state, determining a level of scattering of the light by the liquid based on the degree of polarization of light measured by the polarization detection unit;
determining a level of reflectance and absorption of the light by the liquid based on the intensity of light measured by the image capture device;
acquiring a wavelength and polarization state of light for imaging a structure at least partially obscured, in the visible wavelength range, by the liquid in accordance with the level of scattering, reflectance and absorption of light which has been determined for each of the at least one predetermined wavelength and polarization state.

15) A method of imaging a structure at least partially obscured, in a visible wavelength range, by a liquid, the method comprising:
illuminating, using a light source, the structure with light of a predetermined wavelength and polarization state, the predetermined wavelength and polarization state being acquired by a method according to clause 14; and
generating, using a second image capture device, a second image of the structure, as illuminated by the light source, at the predetermined wavelength.

16) A computer program product comprising instructions which, when implemented by a computer, cause the computer to perform a method according to clause 14 or 15.

Obviously, numerous modifications and variations of the present disclosure are possible in light of the above teachings. It is therefore to be understood that within the scope of the appended claims, the disclosure may be practiced otherwise than as specifically described herein.

In so far as embodiments of the disclosure have been described as being implemented, at least in part, by software-controlled data processing apparatus, it will be appreciated that a non-transitory machine-readable medium carrying such software, such as an optical disk, a magnetic disk, semiconductor memory or the like, is also considered to represent an embodiment of the present disclosure.

It will be appreciated that the above description for clarity has described embodiments with reference to different functional units, circuitry and/or processors. However, it will be apparent that any suitable distribution of functionality between different functional units, circuitry and/or processors may be used without detracting from the embodiments.

Described embodiments may be implemented in any suitable form including hardware, software, firmware or any combination of these. Described embodiments may optionally be implemented at least partly as computer software running on one or more data processors and/or digital signal processors. The elements and components of any embodiment may be physically, functionally and logically implemented in any suitable way. Indeed the functionality may be implemented in a single unit, in a plurality of units or as part of other functional units. As such, the disclosed embodiments may be implemented in a single unit or may be physically and functionally distributed between different units, circuitry and/or processors.

Although the present disclosure has been described in connection with some embodiments, it is not intended to be limited to the specific form set forth herein. Additionally, although a feature may appear to be described in connection with particular embodiments, one skilled in the art would recognize that various features of the described embodiments may be combined in any manner suitable to implement the technique.

1. An apparatus for acquiring a wavelength and polarization state of light for imaging a structure at least partially obscured, in a visible wavelength range, by a liquid, the apparatus comprising:
a light source configured to generate light of a certain wavelength and polarization state, wherein the certain wavelength of light is a wavelength of light within a range of wavelengths outside a visible wavelength range;
a beam splitting element to split light from a target, when the target is illuminated by the light source, along a first path and a second path;
a polarization detection unit arranged on the first path of the beam splitting element and configured to measure a degree of polarization of light;
an image capture device arranged on the second path of the beam splitting element and configured to measure intensity of light at wavelengths within the range of wavelengths outside the visible wavelength range;
and circuitry configured to:
control the light source to generate, in a sequence, light of at least one predetermined wavelength and polarization state to illuminate a liquid;
and, for light of each of the at least one predetermined wavelength and polarization state,
determine a level of scattering of the light by the liquid based on the degree of polarization of light measured by the polarization detection unit;
determine a level of reflectance and absorption of the light by the liquid based on the intensity of light measured by the image capture device;
and wherein the circuitry is further configured to acquire a wavelength and polarization state of light for imaging a structure at least partially obscured, in the visible wavelength range, by the liquid in accordance with the level of scattering, reflectance and absorption of light which has been determined for each of the at least one predetermined wavelength and polarization state.

2. The apparatus according to claim 1, wherein the image capture device is configured to measure intensity of light at wavelengths in the range of 400 nm to 1700 nm.

3. The apparatus according to claim 1, wherein the light source comprises a plurality of light emitting diodes and/or colour filters for generating the sequence of light of at least one predetermined wavelength and polarization state.

4. The apparatus according to claim 1, wherein the circuitry is configured to acquire the wavelength and polarization state of light by determining the wavelength and polarization state of light from amongst the predetermined wavelength and polarization state which provides the minimum scattering, reflectance and absorption of light by the liquid.

5. The apparatus according to claim 1, wherein the light source is arranged on an opposite side of the liquid to the image capture device and the polarization detection unit, and wherein light from the liquid is light transmitted by the liquid from the light source.

6. The apparatus according to claim 1, wherein the light source is arranged on a same side of the liquid to the image capture device and polarization detection unit; wherein the apparatus further comprises a reflection unit submersible in the liquid; and wherein light from the liquid is light reflected from the reflection unit through the liquid from the light source.

7. An apparatus for imaging a structure at least partially obscured, in a visible wavelength range, by a liquid, the apparatus comprising:
a light source configured to illuminate the structure with light of a predetermined wavelength and polarization state, the predetermined wavelength and polarization state being acquired by an apparatus according to claim 1; and
a second image capture device configured to generate a second image of the structure, as illuminated by the light source, at the predetermined wavelength.

8. The apparatus according to claim 7, wherein the light source is further configured to generate light of the visible wavelength region; and wherein the apparatus further comprises:
a third image capture device configured to generate a third image of the structure, as illuminated by the light source, at the visible wavelength range;
and circuitry configured to combine the second image from the second image capture device and the third image from the third image capture device to create an output image of the structure.

9. The apparatus according to claim 8, wherein the circuitry is configured to combine the second image from the second image capture device and the third image from the third image capture device by colouring pixels of the second image based on the pixels of the third image.

10. The apparatus according to claim 8, wherein the circuitry is configured to combine the second image from the second image capture device and the third image from the third image capture device by:
classifying each pixel of the image from each of the second and third image capture device as liquid or non-liquid;
using the pixel from the third image as the corresponding pixel of the output image when the pixel is non-liquid and using the pixel from the second image as the corresponding pixel of the output image when the pixel is classified as liquid;
and colouring the pixels of the output image corresponding to the second image based on the pixels of the third image.

11. The apparatus according to claim 9 or 10, wherein the circuitry is configured to perform the classifying and/or colouring using a machine learning and/or deep learning process.

12. The apparatus according to claim 8, wherein the circuitry is configured to control the intensity of the light source in accordance with an amount of liquid which obscures the structure, and wherein the amount of liquid obscuring the structure is determined in accordance with at least one of the image captured by the second and third image capture device, a signal from an external device and/or an input from a user.

13. An endoscopic device comprising the apparatus according to claims 1 to 6 and/or the apparatus according to claims 7 to 12.

14. A method of acquiring a wavelength and polarization state of light for imaging a structure at least partially obscured, in a visible wavelength range, by a liquid, the method comprising:
generating, using a light source, light of a certain wavelength and polarization state, wherein the certain wavelength of light is a wavelength of light within a range of wavelengths outside a visible wavelength range;
splitting, using a beam splitting element, light from a target, when the target is illuminated by the light source, along a first path and a second path;
measuring a degree of polarization of light using a polarization detection unit arranged on the first path of the beam splitting element;
measuring intensity of light at wavelengths within the range of wavelengths outside the visible wavelength range using an image capture device arranged on the second path of the beam splitting element;
controlling the light source to generate, in a sequence, light of at least one predetermined wavelength and polarization state to illuminate a liquid;
and, for light of each of the at least one predetermined wavelength and polarization state,
determining a level of scattering of the light by the liquid based on the degree of polarization of light measured by the polarization detection unit;
determining a level of reflectance and absorption of the light by the liquid based on the intensity of light measured by the image capture device;
acquiring a wavelength and polarization state of light for imaging a structure at least partially obscured, in the visible wavelength range, by the liquid in accordance with the level of scattering, reflectance and absorption of light which has been determined for each of the at least one predetermined wavelength and polarization state.

15. A method of imaging a structure at least partially obscured, in a visible wavelength range, by a liquid, the method comprising:
illuminating, using a light source, the structure with light of a predetermined wavelength and polarization state, the predetermined wavelength and polarization state being acquired by a method according to claim 14; and
generating, using a second image capture device, a second image of the structure, as illuminated by the light source, at the predetermined wavelength.
